(19) United States
(12) Patent Application Publication
(10) Pub. No.: US 2022/0183722 A1
(43) Pub. Date: Jun. 16, 2022

(54) PEDICLE TRACT STABILIZATION SYSTEM

(71) Applicant: Sanjay Amarasinghe, Singapore (SG)

(72) Inventor: Sanjay Amarasinghe, Singapore (SG)

(21) Appl. No.: 17/117,468

(22) Filed: Dec. 10, 2020

(51) Int. Cl. A61B 17/70; A61B 17/86

(52) U.S. Cl. CPC A61B 17/7032; A61B 2017/867; A61B 17/8665; A61B 17/863

(57) ABSTRACT

The present application relates to a pedicle tract stabilization system comprising a bone anchor and a fixation mechanism. The bone anchor is used for stabilizing vertebra during surgical instrumentation of the spine; and the fixation mechanism is used for fixing a bone anchor during the surgical instrumentation. The bone anchor comprises a gripping mechanism for securing the bone anchor to the vertebra cortex and a central body for coupling the gripping mechanism. The fixation mechanism comprises a frame movably attached to the bone anchor. In addition to the bone anchor and the fixation mechanism, the pedicle tract stabilization system may further comprise an external clamping mechanism and a linking mechanism.

### CROSS REFERENCE TO RELATED APPLICATIONS

Not Applicable.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND

### Technical Field

The present application relates to a pedicle tract stabilization system for fixing a pedicle screw into a vertebra. The pedicle tract stabilization system is also known as a pedicle tract stabilization device or apparatus. More particularly, the present application relates to a bone anchor of the pedicle tract stabilization system for stabilizing the vertebra during surgical instrumentation of the spine as well as a fixation mechanism of the pedicle tract stabilization system for fixing the bone anchor in place during the surgical instrumentation.

### Prior Art

There have been revolutionary advancements in spinal neuronavigational technology in the last few decades. Computed Tomography (CT) guided imaging techniques and integration with robotics have optimized accuracy of pedicle screw placement. Currently, methods of eliminating motion artefacts into a navigational system are available; However, the current methods cannot prevent motion occurring during pedicle screw preparation and insertion. Furthermore, cancellation of the motion artefacts does not ensure that an intended screw trajectory is preserved. In fact, a real screw trajectory may be deviated from the intended screw trajectory and may require revisions during pedicle screw insertion.

In order to resolve these problems, the present application introduces a pedicle tract stabilization system for preparing and inserting a pedicle screw in a vertebra, which would have applications in spinal operations, including minimally invasive surgery (MIS). The pedicle tract stabilization system also inherently enables an automated or manually operated drill/screwdriver to detect a potential breach in the pedicle cortex (e.g. the breach is determined by indirect measures of a change in resistance of the pedicle cortex), thereby informing the surgeon to revise the current trajectory. Furthermore, the proposed pedicle tract stabilization system could also provide a channel for insertion of an ultrasound device to visualize walls of the pedicles which can help to optimize the planned trajectory for pedicle tract preparation and screw insertion and potentially eliminate the need for intraoperative radiation required in neuronavigational techniques. In addition, the proposed pedicle tract stabilization system may also have other advantages, including being a lightweight system, easy to operate and cost-effective.

### BRIEF SUMMARY OF NON-LIMITING EXEMPLARY EMBODIMENT(S) OF THE PRESENT DISCLOSURE

In view of the foregoing background, it is therefore an object of the non-limiting exemplary embodiment(s) to provide a pedicle tract stabilization system. These and other objects, features, and advantages of the non-limiting exemplary embodiment(s) are provided by a first aspect, wherein the present application discloses a bone anchor for stabilizing the vertebra when the pedicle screw and preparation instruments (such as awl, pedicle probe and tap screw) are inserted into the vertebra. The bone anchor comprises a gripping mechanism for securing the bone anchor to the vertebra and a central body for coupling the gripping mechanism. In particular, the gripping mechanism is configured to form an aperture (also known as lumen) for the pedicle screw and other instruments to pass through the gripping mechanism. For example, an awl is attached at an end of a stylet or a slender probe which is inserted through the aperture to reach a pre-determined location in the vertebra, such as a drilled facet, an inferior articular process or a lateral mass.

The gripping mechanism optionally comprises a tapered screw with a plurality of screw threads for securing the bone anchor to the surrounding vertebral cortex. The screw threads may have various depths to provide better purchase with the vertebral cortex surrounding the tapered screw for reducing or even eliminating any likelihood of pullout and advancement of the bone anchor from a fixed position in the vertebral cortex.

The tapered screw optionally has an inner side with an inner angle and an outer side with an outer angle. The inner side and the outer side face the aperture and the vertebral cortex, respectively. The screw threads are optionally coupled to the outer side of the gripping means for providing better anchorage with the surrounding vertebral cortex. In particular, the inner angle is larger than the outer angle, since the inner angle is designed to accommodate a wide range of screw trajectories while the outer angle aims to facilitate acceptance of the bone anchor when the bone anchor is screwed into a decorticated edge of the drilled facet, the inferior articular process or the lateral mass.

The inner side and the outer side of the tapered screw are configured to form a sharp edge for cutting through underlying cancellous bone of the vertebral cortex for preparing a screw trajectory in the vertebra. The profile of the tapered screw can either be a conical-cutting configuration or crown-cutting configuration. The conical-cutting configuration has a fixed inner angle and thus a fixed screw depth which permits only a maximum inner angle for the screw to rest. While the crown-cutting configuration has a variable inner angle which provides a greater degree of angulation for the screw to rest and also provides a greater screw depth. Therefore, the crown-cutting configuration is more advantageous as it provides a wider range of possible screw trajectories and also greater screw depth for better integration into surrounding cancellous bones.

The central body may further comprise a base coupled to the tapered screw for mechanically supporting the tapered screw. The base is opposed to the sharp edge of the tapered screw. The base is also used to couple the tapered screw to other parts of the pedicle tract stabilization system. The bone anchor optionally further comprises an adjustable head movably coupled to the base. In some implementations, the base has a first hinge or a first movable joint movably coupled to the adjustable head for enabling greater rotation of the adjustable head. Therefore, the first hinge allows the pedicle screw or other surgical instruments to be orientated in a certain direction through the aperture. The adjustable head is configured to form an internal cavity aligned with the aperture for the pedicle screw or other surgical instruments to pass through the adjustable head, the base and the tapered screw.

Within the internal cavity and the aperture, the bone anchor is optionally configured to form a screw trajectory along which the pedicle screw or other surgical instruments are inserted into the pedicle cortex. In particular, the screw trajectory is configured to adjust in a certain range in the internal cavity and the aperture. The certain range is determined by two factors, firstly widths of the internal cavity and the aperture; and secondly the inner angle of the tapered screw. Hence, the wider the internal cavity and the aperture are, and the larger the inner angle is, the larger the range of available screw trajectories there will be.

The adjustable head is configured to form a side aperture for a bar to laterally pass through. In this way, multiple bone anchors loaded at different vertebral levels on one side of the spinal column are assembled together by inserting the bar laterally through their side apertures for thereby forming a stable screw-rod fixation construct. In particular, the bar may have a curved configuration for matching a curved profile of the spinal column. If one of the bone anchors is accidently not stabilized, the rest bone anchors could help fix the unstable bone anchor via the bar.

The adjustable head optionally comprises a threaded end for coupling to a fixation mechanism. For example, the adjustable head has a threaded end opposed to the tapered screw; while the fixation mechanism has complementary threads matching the threaded end; and thus the adjustable head is firmly assembled with the fixation mechanism at the threaded end.

The bone anchor may further comprise a stylet coupled to the base for stabilizing the bone anchor when the bone anchor is screwed into decorticated facet in the vertebral cortex. In some implementations, the stylet has a first stylet arm and a second stylet arm coupled to a left side and a right side of the base of the bone anchor, respectively. For example, the base has a first hole and a second hole on the left side and the right side for receiving the first stylet arm and the second stylet arm, respectively.

The bone anchor may further comprise a locking nut superimposed onto the bar within the adjustable head for securing the bar to the bone anchor. The locking nut (also known as locknut, lock nut, self-locking nut or stiff nut) could resist loosening under vibrations and torque when the bone anchor is inserted into the vertebra vortex. In some implementations, the locking nut comprises a prevailing torque nut or an elastic stop unit of which some portion deforms elastically to provide locking action.

The pedicle screw optionally comprises a polyaxial screw which is easily aligned correctly with the screw trajectory, since the polyaxial screw could be adjusted to multiple axes directing to the screw trajectory without hindrance. The polyaxial screw may further comprise a polyaxial screw base configured to load on the base; and a screw shaft movably coupled to the screw chassis. In particular, the screw shaft is configured to form an acute angle with the screw base.

As a second aspect, the present application discloses a fixation mechanism for fixing a bone anchor. The fixation mechanism comprises a frame coupled to the bone anchor. In particular, the frame is configured to form an internal passage for a pedicle screw and other surgical instruments (such as pedicle tract preparation instruments) to pass through. Therefore, the frame, the adjustable head, the base and the tapered screw are configured to form a through channel for the pedicle screw and other surgical instruments to pass through. In some implementations, the frame comprises a flute having a hollow cylindrical configuration which matches the adjustable head in size.

The fixation mechanism may further comprise an internal stabilizing component (also known as internal stabilizer) coupled within the frame for minimizing buckling of the bone anchor in operation by allowing rotatory movement of the bone anchor only. Therefore, excessive motions of surgical instruments (i.e. pedicle probe, tap screw and pedicle screw) are minimized or even eliminated in automated or manually-performed stages of pedicle tract preparation and screw insertion when they are advanced through the cancellous medium of the pedicle and vertebral body. In some implementations, the internal stabilizer is coupled within a mid-section of the frame for minimizing or even eliminating buckling of surgical instruments during the stages of pedicle tract preparation and screw insertion. The fixation mechanism may further comprise an inset coupled within the frame for supporting the internal stabilizer. The inset has a small size than the internal stabilizer such that the inset would not block the surgical instruments to advance within the internal passage of the frame.

The internal stabilizer optionally has a plurality of stabilizing teeth which complement the bolt threads of a threaded bolt attached to an expanded portion of the surgical instruments for stabilizing their advancement through the pedicle and vertebral body. In other words, the internal stabilizing component and the surgical instruments are tightly engaged together by the stabilizing teeth and the bolt teeth for resisting external turbulences during automated or manually-performed stages of spinal instrumentation.

As a third aspect, the present application discloses a pedicle tract stabilization system. The pedicle tract stabilization system comprises one or more bone anchors for stabilizing one or more vertebra; and a fixation mechanism movably attached to the one or more bone anchors. In particular, the bone anchors and the fixation mechanism are configured to form a through channel for a pedicle screw and surgical instruments (such as pedicle tract preparation instruments) to pass through.

The pedicle tract stabilization system may further comprise an external clamping mechanism for clamping the bone anchor and the fixation mechanism to a stationary object (such as a surgical table); and a linking mechanism for movably coupling the fixation mechanism and the external clamping mechanism. The stationary object is firmly secured to the ground for preventing any motion of the pedicle tract stabilization system during surgery. In some implementations, the stationary object comprises a surgical table where a patient to be operated on during surgery is also laid.

The linking mechanism may further comprise a circular frame coupled to the fixation mechanism, an external arm having a proximal end and a distal end and a cuboidal clamp. The circular frame and the cuboidal clamp are movably coupled (such as via a hinge mechanism) to the proximal end and the distal end of the external arm, respectively. The circular frame would pass through the channel for stabilizing the fixation mechanism. The cuboidal clamp is movably coupled to the distal end and the external clamping mechanism for coupling the linking mechanism and the external clamping mechanism. In some implementations, the external arm further comprises a first sub-arm and a second sub-arm adjoined by a sliding-hinge mechanism for enabling the external arm to extend or contract along a single axis. In some implementations, the cuboidal clamp may further comprise a ball and a socket joint movably coupled together for providing a greater degree of maneuverability. For example, the ball could rotate substantially within the socket joint.

In some implementations, the external clamping mechanism comprises a supporting means (such as a rod) movably coupled to the cuboidal clamp and a clamping means coupled to the supporting means and the stationary object. For example, the clamping means comprises a table clamp hinged to the stationary object (such as the surgical table) for easy operation during the surgery. Therefore, the pedicle tract stabilization system as a whole would serve to annul any motion induced during the surgery and also preserve the pedicle tract trajectory at all stages of the surgery by locking the bone anchor with the fixation mechanism and clamping system.

The pedicle tract stabilization system may further comprise a reference frame for accurately locating and orientating the bone anchor head and an attached frame. For example, the reference frame is loaded onto an adjacent vertebra around a targeted vertebra onto which the bone anchor and frame would be secured. An external sensor detects the relative positions of optical (such as reflective) markers located on both the reference frame and the frame (such as flute), thereby enabling them to be mapped out in 3D space. The mappings can be superimposed on an intraoperative CT image of the spine which can then be used to correctly orientate the bone anchor and attached frame (such as flute) in a real-time trajectory with the planned trajectory from the CT.

As a fourth aspect, the present application discloses a surgical process with the pedicle tract stabilization system. Firstly, a patient is positioned and prepped in a standard fashion for pedicle screw fixation procedure. Pedicle screws may be inserted under open or MIS techniques. Once facet joints are exposed, a facetectomy is performed using bone rongeurs or a high-speed drill. A reference frame is attached to a specified spinous process of a vertebra above or below the levels of intended spinal fixation. An intraoperative computed tomography (CT) scan is performed to serve as a neuronavigational guide.

Secondly, a bone anchor with suitable dimensions to a specific spinal level is loaded onto a frame with an egg handle attached thereto. The bone anchor is screwed into the facetectomised cortex on one side of a specified vertebra until good purchase is achieved. The frame is then stabilized with an external clamping mechanism in a specific position and orientation, determined either using ultrasound techniques or neuronavigational guidance. For example, the ultrasound techniques use a tubular ultrasound scanner which can be passed through the central axis along length of the frame (such as the flute) into the aperture of the bone anchor. During manually positioning the frame (such as the flute), an ultrasound transducer at the tip of the device would scan the contour of the pedicle and thus help to optimize the final position of the frame (such as the flute). Alternatively, the frame (such as the flute) may be correctly positioned by neuronavigational guidance by aligning the real-time trajectory that is based on neuronavigational markers located on the frame (such as the flute) in relation to the reference frame on a spinous process of an adjacent vertebra with the planned trajectory. The position of the frame (such as the flute) is adjusted by theta degrees in a cross-sectional view and alpha degrees in a lateral view such that the real time trajectory coincides with the planned trajectory determined using the intraoperative computed tomography. Therefore, the neuronavigational guidance for positioning the frame (such as the flute) is used as an alternative technique to the ultrasound techniques.

Once the frame (such as the flute) is correctly positioned and orientated, the entire pedicle tract stabilization system is locked. The pedicle is prepared with a pedicle probe followed by a tap screw or reamer. The pedicle screw is then inserted. In both pedicle preparation and screw insertion processes, the surgical instruments are stabilized by the complementary teeth of the threaded bolt of the surgical instrument and the internal stabilizer contained within the mid-section of the frame for minimizing micro-movements and also maintaining a same trajectory.

Thirdly, the processes above are repeated for other vertebral levels and also on the contralateral side of the spine. The frame and external clamping mechanism are removed. Once all the pedicle screws are inserted into the vertebra, a rod is passed through the apertures on both sides of each bone anchor, thereby interconnecting the bone anchors of adjacent vertebral levels. Finally, locking nuts are applied to the adjustable head of each bone anchor in order to secure the rod in place.

Therefore, the pedicle tract stabilization system can stabilize a specified vertebra during pedicle screw preparation and insertion; preserve a planned trajectory during all stages of pedicle screw preparation and insertion in order to optimize the accuracy of pedicle screw placement; eliminate motion artefact and hence providing accurate visual feedback to a surgeon; discard the need for methods of eliminating computational artefact employed by other spinal neuronavigational systems; and minimize trajectory and screw revision, which will be both cost effective and less time consuming.

Furthermore, an automated drill screwdriver system may be utilized in conjunction with the bone anchor and external clamping system of the subject application. The automated drill screwdriver system may be further coupled to an integrated pressure-sensing mechanism to detect potential pedicle cortex breach. The automated drill screwdriver system can be used by including an external rectangular frame with two sets of perpendicularly running platforms joined to the surgical table. This frame system with two sets of perpendicularly running platforms is operated robotically and thus transports the automated drill-screwdriver system between a box-set of pedicle screws and each individual frame (such as the flute). The movements of the motorized platform and alignment of the automated drill-screwdriver system are stereotactically governed by the pre-determined trajectories from the frame (such as the flute) and the position of the box-set of pedicle screws relative to the reference frame. The reference frame serves as an origin with a (0,0,0) landmark for neuronavigation based on a 3D Cartesian coordinate system (x,y,z).

There has thus been outlined, rather broadly, the more important features of non-limiting exemplary embodiment(s) of the present disclosure so that the following detailed description may be better understood, and that the present contribution to the relevant art(s) may be better appreciated. There are additional features of the non-limiting exemplary embodiment(s) of the present disclosure that will be described hereinafter and which will form the subject matter of the claims appended hereto.

### BRIEF DESCRIPTION OF THE NON-LIMITING EXEMPLARY DRAWINGS

The novel features believed to be characteristic of non-limiting exemplary embodiment(s) of the present disclosure are set forth with particularity in the appended claims. The non-limiting exemplary embodiment(s) of the present disclosure itself, however, both as to its organization and method of operation, together with further objects and advantages thereof, may best be understood by reference to the following description taken in connection with the accompanying drawings in which:

FIG. 1 illustrates (a) a cross-sectional view and (b) an aerial view of a bone anchor;

FIG. 2 illustrates (a) a conical cutting profile for a conical tapered screw; and (b) a crown cutting profile for a crown tapered screw;

FIG. 3 illustrates (a) a cross-sectional view and (b) a lateral view of the bone anchor before an inserted bar; and (c) a cross-sectional view and (d) a lateral view of the bone anchor after the inserted bar;

FIG. 4 illustrates an enlarged cross-sectional view of the bone anchor;

FIG. 5 illustrates a cross-sectional view of the bone anchor with a style;

FIG. 6 illustrates a cross-sectional view of a range of trajectories available within the bone anchor;

FIG. 7 illustrates (a) a cross-sectional view of a left leaning trajectory when the bone anchor is rotated to the left side; and (b) a cross-sectional view of a right leaning trajectory when the bone anchor is rotated to the right side;

FIG. 8 illustrates a cross-sectional view of the bone anchor loaded with a polyaxial screw;

FIG. 9 illustrates (a) a cross-sectional view of the bone anchor before purchase with surrounding cortex; and (b) a cross-sectional view of the bone anchor after purchase with the surrounding cortex;

FIG. 10 illustrates a cross-sectional view of bone anchor secured within the surrounding cortex;

FIG. 11 illustrates (a) a cross-sectional view of a frame having two insets; and (b) a cross-sectional view of a frame having the insets and an internal stabilizing component;

FIG. 12 illustrates a cross-sectional view of the insets and the internal stabilizing component to the bone anchor in five subsequent stages of pedicle preparation and screw insertion processes;

FIG. 13 illustrates another cross-sectional view of the insets and the internal stabilizing component to the bone anchor in five subsequent stages of pedicle preparation and screw insertion processes;

FIG. 14 illustrates another cross-sectional view of the insets and the internal stabilizing component to the bone anchor in five subsequent stages of pedicle preparation and screw insertion processes;

FIG. 15 illustrates another cross-sectional view of the insets and the internal stabilizing component to the bone anchor in five subsequent stages of pedicle preparation and screw insertion processes;

FIG. 16 illustrates another cross-sectional view of the insets and the internal stabilizing component to the bone anchor in five subsequent stages of pedicle preparation and screw insertion processes;

FIG. 17 illustrates (a) a cross-sectional view of a linking mechanism; and (b) an aerial view of the linking mechanism;

FIG. 18 illustrates a cross-sectional view of the bone anchor secured to vertebra cortex;

FIG. 19 illustrates a cross-sectional view of adjustment of a real-time trajectory to coincide with a planned trajectory;

FIG. 20 illustrates a lateral view of the adjustment of real-time trajectory to coincide with planned trajectory;

FIG. 21 illustrates a cross-sectional view of an external clamping mechanism for stabilizing the bone anchor to trunk;

FIG. 22 illustrates a cross-sectional view of a series of bone anchors connected together by a bar; and

FIG. 23 illustrates an aerial view of the series of bone anchors connected together by the bar.

Those skilled in the art will appreciate that the figures are not intended to be drawn to any particular scale; nor are the figures intended to illustrate every non-limiting exemplary embodiment(s) of the present disclosure. The present disclosure is not limited to any particular non-limiting exemplary embodiment(s) depicted in the figures nor the shapes, relative sizes or proportions shown in the figures.

### DETAILED DESCRIPTION OF NON-LIMITING EXEMPLARY EMBODIMENT(S) OF THE PRESENT DISCLOSURE

The present disclosure will now be described more fully hereinafter with reference to the accompanying drawings, in which non-limiting exemplary embodiment(s) of the present disclosure is shown. The present disclosure may, however, be embodied in many different forms and should not be construed as limited to the non-limiting exemplary embodiment(s) set forth herein. Rather, such non-limiting exemplary embodiment(s) are provided so that this application will be thorough and complete, and will fully convey the true spirit and scope of the present disclosure to those skilled in the relevant art(s). Like numbers refer to like elements throughout the figures.

The illustrations of the non-limiting exemplary embodiment(s) described herein are intended to provide a general understanding of the structure of the present disclosure. The illustrations are not intended to serve as a complete description of all of the elements and features of the structures, systems and/or methods described herein. Other non-limiting exemplary embodiment(s) may be apparent to those of ordinary skill in the relevant art(s) upon reviewing the disclosure. Other non-limiting exemplary embodiment(s) may be utilized and derived from the disclosure such that structural, logical substitutions and changes may be made without departing from the true spirit and scope of the present disclosure. Additionally, the illustrations are merely representational are to be regarded as illustrative rather than restrictive.

One or more embodiment(s) of the disclosure may be referred to herein, individually and/or collectively, by the term “non-limiting exemplary embodiment(s)” merely for convenience and without intending to voluntarily limit the true spirit and scope of this application to any particular non-limiting exemplary embodiment(s) or inventive concept. Moreover, although specific embodiment(s) have been illustrated and described herein, it should be appreciated that any subsequent arrangement designed to achieve the same or similar purpose may be substituted for the specific embodiment(s) shown. This disclosure is intended to cover any and all subsequent adaptations or variations of other embodiment(s). Combinations of the above embodiment(s), and other embodiment(s) not specifically described herein, will be apparent to those of skill in the relevant art(s) upon reviewing the description.

References in the specification to “one embodiment(s)”, “an embodiment(s)”, “a preferred embodiment(s)”, “an alternative embodiment(s)” and similar phrases mean that a particular feature, structure, or characteristic described in connection with the embodiment(s) is included in at least an embodiment(s) of the non-limiting exemplary embodiment(s). The appearances of the phrase “non-limiting exemplary embodiment” in various places in the specification are not necessarily all meant to refer to the same embodiment(s).

Directional and/or relationary terms such as, but not limited to, left, right, nadir, apex, top, bottom, vertical, horizontal, back, front and lateral are relative to each other and are dependent on the specific orientation of an applicable element or article, and are used accordingly to aid in the description of the various embodiment(s) and are not necessarily intended to be construed as limiting.

If used herein, “about,” “generally,” and “approximately” mean nearly and in the context of a numerical value or range set forth means±15% of the numerical.

If used herein, “substantially” means largely if not wholly that which is specified but so close that the difference is insignificant.

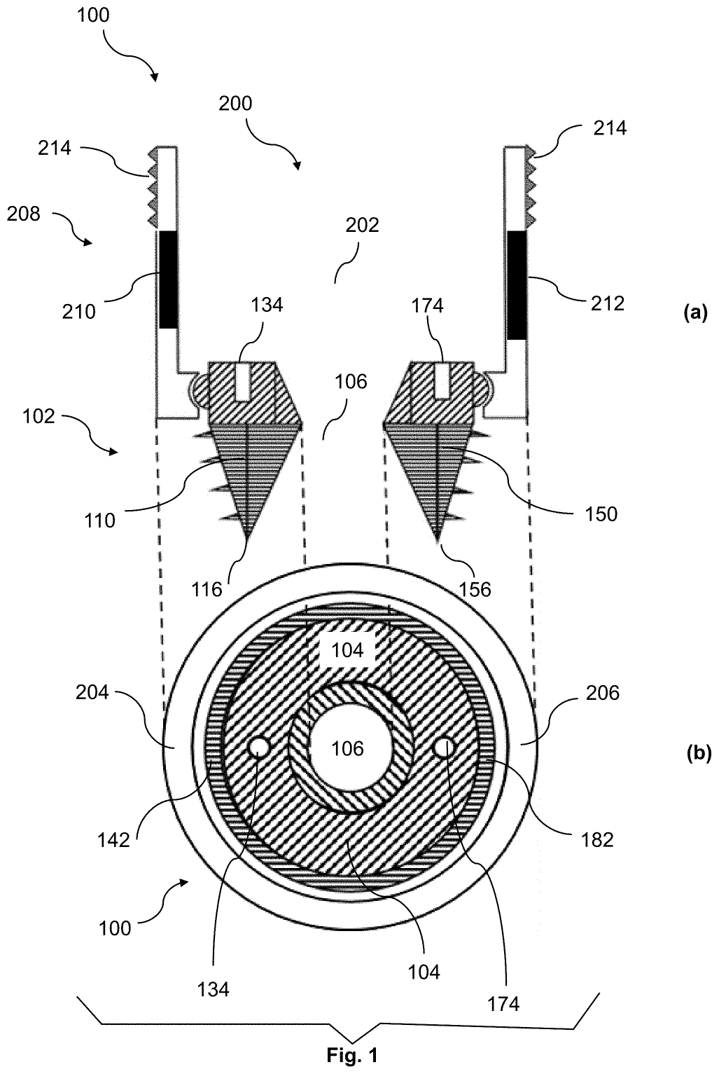

FIG. 1 illustrates (a) a cross-sectional view and (b) an aerial view of a bone anchor 100. The bone anchor 100 has a conical tapered screw 102 and a base (also known as central body or main body) 104 as a gripping mechanism and a central body, respectively. The conical tapered screw 102 is used to secure the bone anchor 100 to surrounding vertebral cortex (not shown). The base 104 is used to support the conical tapered screw 102 which is connected beneath the base 104. Both the conical tapered screw 102 and the base 104 have ring structures with an aperture 106 formed at the center of a concentric configuration. The base 104 comprises a left hinge 132 and a right hinge 172 movably attached to a left concave structure 204 and a right concave structure 206 of an adjustable head 200 of the bone anchor 100, respectively. Therefore, the conical tapered screw 102 is movably coupled to the adjustable head 200. In addition, the base 104 comprises a left hole 134 and a right hole 174 aligned with the left hinge 142 and the right hinge 182 respectively, both of which are exposed outside the base 104.

The conical tapered screw 102 is imaginarily divided into a left portion 110 and a right portion 150 in the cross-sectional view. Accordingly, the base 104 is also imaginarily divided into a left portion 140 and a right portion 180 in the cross-sectional view for connecting the left portion 110 and the right portion 150 of the conical tapered screw 102, respectively. It is clearly shown that the conical tapered screw 102 has a tapering inner profile and a tapering outer profile from the top to the bottom; and a sharp edge (not shown) is formed at the bottom. As a result, both the left portion 110 and the right portion 150 have a triangle shape in the cross-sectional view; and the sharp edge is reduced to a left sharp point 116 and a right sharp point 156 at the left portion 110 and the right portion 150, respectively. The base 104 has an expanding inner profile and a vertical outer profile from the top to the bottom. In other words, the left portion 140 has a left inner face 146 opposed from the left hinge 142; and the right portion 180 also has a right inner face 186 opposed from the right hinge 182. As a result, both the left portion 140 and the right portion 180 have a trapezoid shape in the cross-sectional view. In particular, an internal cavity 202 is formed in the adjustable head 200 and the internal cavity 202 further forms a through channel with the aperture 106 in a vertical direction for the pedicle screw to pass through the bone anchor 100. A side aperture 208 is also formed in the adjustable head 200 in a lateral direction perpendicular to the internal cavity 202. In the cross-sectional view, the side aperture 208 is shown as a left aperture 210 and a right aperture 212 above the left concave structure 204 and the right concave structure 206, respectively. A rod (not shown) may pass through the hollow hole 208 for connecting multiple bone anchors 100 in series. In addition, the adjustable head 200 has a threaded end 210 opposed to the concave structures 204, 206 for connecting to a fixation mechanism (not shown).

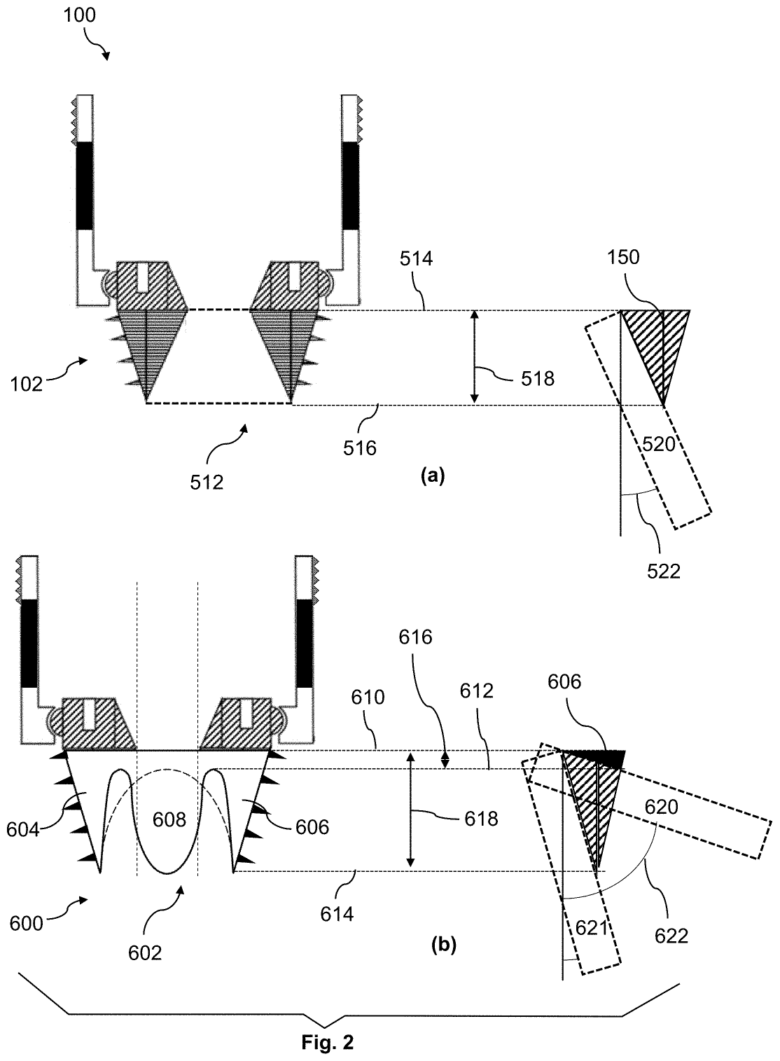

FIG. 2(a) illustrates a conical cutting profile 512 (shown in dash line) for the conical tapered screw 102. The conical cutting profile 512 has a trapezoid shape with an upper cutting line 514 and a lower cutting line 516 which are kept constant and parallel; and thus a distance 518 between the upper cutting line 514 and the lower cutting line 516 is kept unchanged for the conical cutting profile 512. A dash square shows a planned trajectory 520 in contact with the right portion of the conical tapered screw 150. A trajectory angle 522 is used to describe an angle between the vertical direction and the planned trajectory 520. The trajectory angle 522 is adjustable in a range of 5 to 15 degrees. FIG. 2(b) shows a crown tapered screw 600 in replacement of the conical cutting profile 512. The crown tapered screw 600 has a left portion 604, a right portion 606 and a middle portion 608 coupled together. A crown cutting profile 602 (shown in dash line) is also illustrated for the crown tapered screw 600. In contrast to the conical cutting profile 512, the crown cutting profile 602 has an arch shape with an upper cutting line 610, a middle cutting line 612 and a bottom cutting line 614. Therefore, a first distance 616 is created between the upper cutting line 610 and the middle cutting line 612; and a second distance 618 is also created between the middle cutting line 612 and the bottom cutting line 614. In accordance with the first distance 616 and the second distance 618, two dash squares show a first planned trajectory 620 and a second planned trajectory 621 in contact with the right portion of the crown tapered screw 600, respectively. A trajectory angle 622 is used to describe an angle between the vertical direction and the planned trajectories 620, 621. The trajectory angle 622 is adjustable in a range of 5 to 45 degrees.

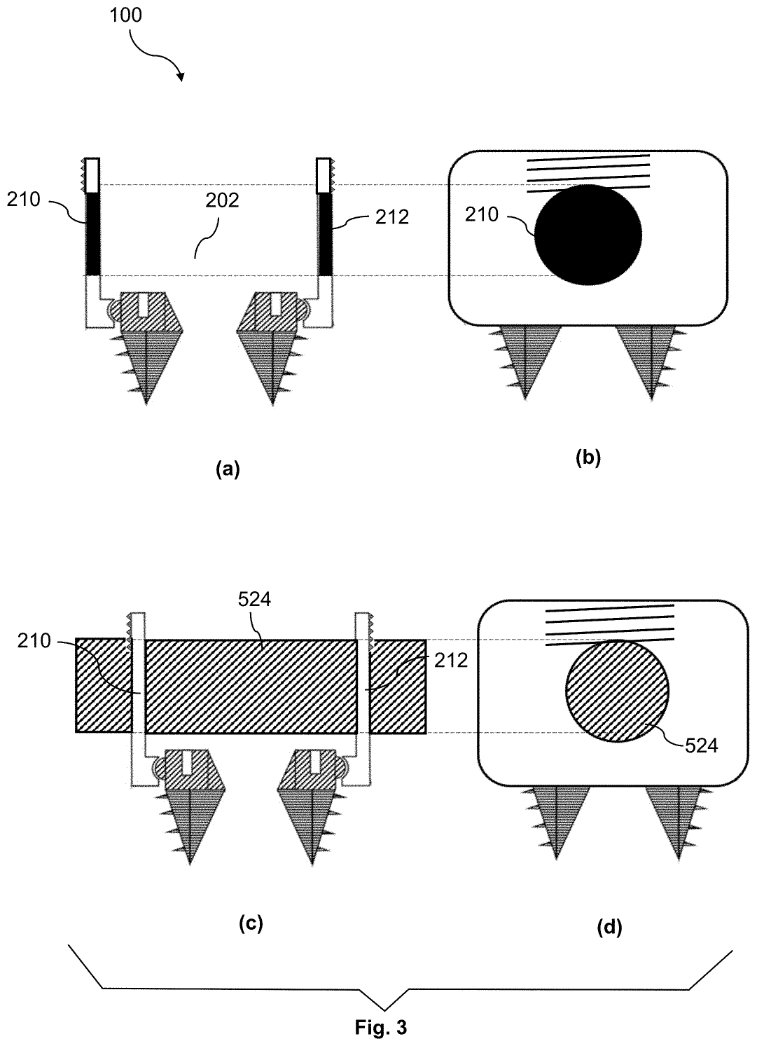

FIG. 3(a) illustrates a cross-sectional view and FIG. 3(b) illustrates a lateral view of the bone anchor 100 before a bar 524 is inserted into the bone anchor 100 laterally. FIG. 3(c) illustrates a cross-sectional view and FIG. 3(d) illustrates a lateral view of the bone anchor 100 after the bar 524 is inserted through the left aperture 210, the internal cavity 202 and the right aperture 212. In a same manner, multiple bone anchors 100 are coupled together by inserting the bar 524 through the bone anchors 100 laterally.

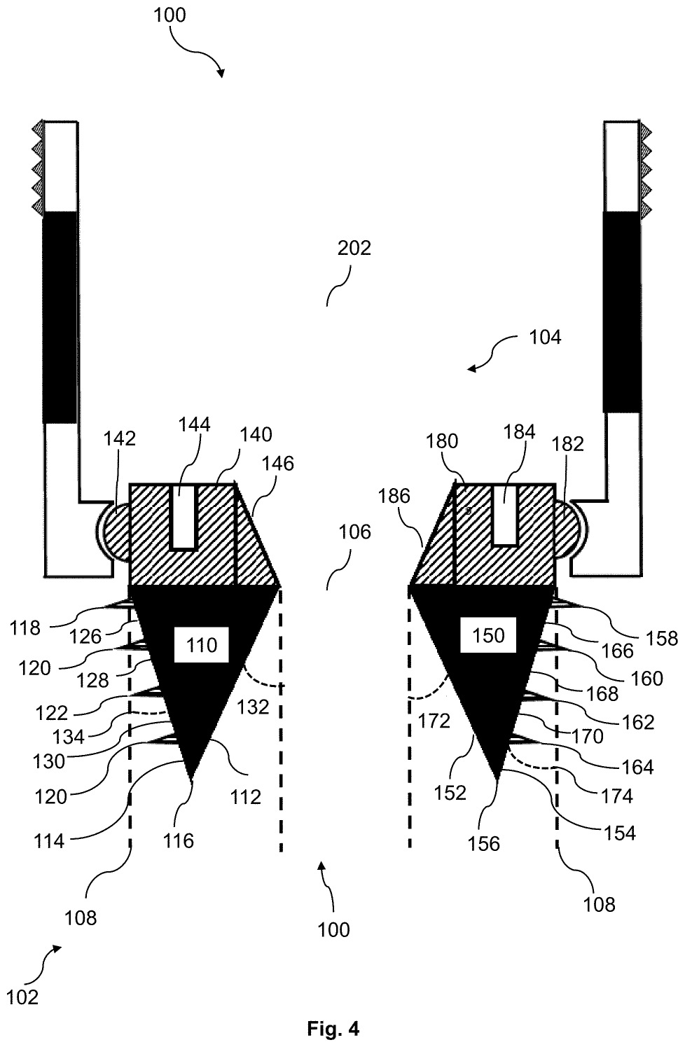

FIG. 4 illustrates an enlarged cross-sectional view of the bone anchor 100. The conical tapered screw 102 has four screw threads surrounding its outer surface. In the cross-sectional view, it is shown that the left portion 110 has a triangular configuration with a left inner side 112 and a left outer side 114, both of which form a left sharp edge 116 for drilling through the vertebra cortex. The left portion 110 also has four screw tips as the four screw threads reduced to the cross-sectional view. The four screw tips are coupled on the left outer side 114 for securing to the surrounding vertebra cortex, i.e. a first left screw tip 118, a second left screw tip 120, a third left screw tip 122 and a fourth left screw tip 124 approaching the left sharp point 116. To provide better purchase with the surrounding vertebra cortex, the left screw tips 118-124 may have various depths which may vary in a range of 1 millimeter (mm) to 4 millimeters (mm), depending on a specific bone of the spinal column the bone anchor 100 is applied to. The left screw tips 118-124 may be flexibly distributed on the left outer side 114. As shown in FIG. 4, the left screw threads 118-124 has a first left distance 126 between the first left screw tip 118 and the second left screw tip 120, a second left distance 128 between the second left screw tip 120 and the third left screw tip 122, and a third left distance 130 between the third left screw tip 122 and the fourth left screw tip 124. The distances 126-130 may vary in a range of 0.5 millimeter (mm) to 3 millimeters (mm), depending on a specific bone of the spinal column the bone anchor 100 is applied to, as well as a specific level of spinal column the surgical operation is conducted on. The left inner side 112 and the left outer side 114 form a left inner angle 132 and a left outer angle 134 in respect to a vertical direction 108 (shown as dash lines), respectively. In particular, the left inner angle 132 is larger than the left outer angle 134 for accommodating a wide range of screw trajectories.

Similarly, the right portion 150 also has a triangular configuration in the cross-sectional view with a right inner side 152 and a right outer side 154, both of which forms a right sharp point 156. The right portion 150 also has a four right screw tips 158-164 coupled on the right outer side 154. To provide better purchase with the surrounding vertebra cortex, the right screw tips 158-164 may also have various depths which may vary in a range of 1 millimeter (mm) to 4 millimeters (mm), depending on a specific bone of the spinal column the bone anchor 100 is applied to. The right screw threads 158-164 may be flexibly distributed on the left outer side 154. As shown in FIG. 4, the right screw threads 158-164 has a first right distance 166 between the first right screw tip 158 and the second right screw tip 160, a second right distance 168 between the second right screw tip 160 and the third right screw tip 162, and a third right distance 170 between the third right screw tip 162 and the fourth right screw tip 164. The distances 166-170 may vary in a range of 0.5 millimeter (mm) to 3 millimeters (mm), depending on a specific bone of the spinal column the bone anchor 100 is applied to, as well as a specific level of spinal column the surgical operation is conducted on. The right inner side 152 and the right outer side 154 also form a right inner angle 172 and a right outer angle 174 in respect to the vertical direction 108 (shown as dash lines), respectively. The right inner angle 172 is also larger than the right outer angle 174. The aperture 106 is formed between the left inner side 112 and the right inner side 152 for a pedicle screw to pass through.

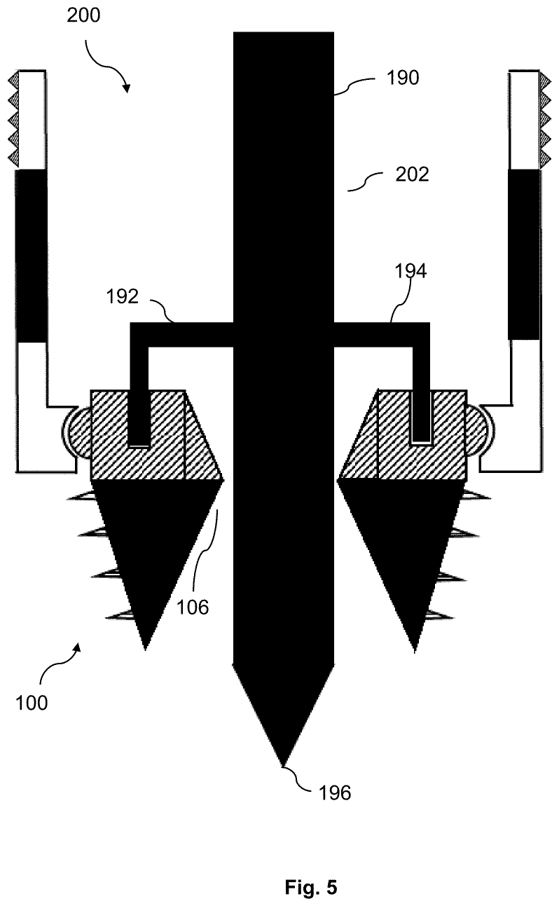

FIG. 5 illustrates a cross-sectional view of the bone anchor 100 with a stylet 190. The stylet 190 is inserted through the internal cavity 202 and the aperture 106 for reaching out of the adjustable head 200 and the tapered screw 102 in sequence. The stylet 190 comprises a first stylet arm 192 and a second stylet arm 194 inserted into the left hole 144 and the right hole 184 of the base 104 respectively. As a result, the stylet 190 stabilizes the bone anchor 100 when the bone anchor 100 is screwed into the surrounding vertebra cortex. In addition, the stylet 190 comprises a sharp end 196 to provide better cutting quality through the vertebral cancellous bone underlying the shaved vertebral cortex.

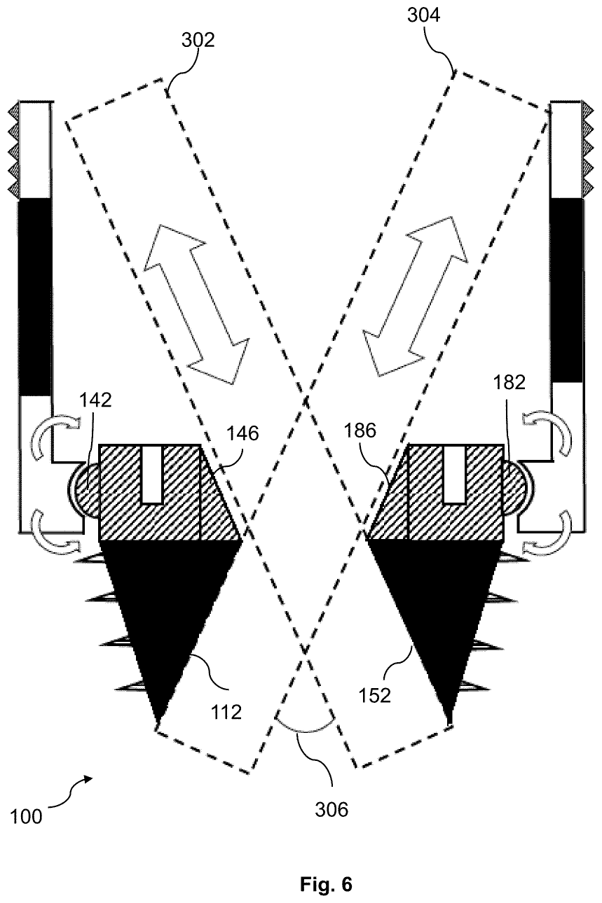

FIG. 6 illustrates a cross-sectional view of a range of trajectories 300 available within the bone anchor 100 when the bone anchor 100 is secured with the surrounding vertebral cortex. The trajectory 300 would be inserted into the internal cavity 202 and the aperture 106. A left leaning trajectory 302 (shown in dash line) represents a left limitation for the trajectory 300 due to the left inner face 146 of the left portion 140 of the base 104 and the right inner side 152 of the right portion 150 of the tapered screw 102. Similarly, a right leaning trajectory 304 (shown in dash line) represents a right limitation for the trajectory 300 due to the right inner face 186 of the right portion 180 of the base 104 and the left inner side 112 of the left portion 110 of the tapered screw 102. The left leaning trajectory 302 and the right leaning trajectory 304 form a trajectory range 306. The larger the trajectory range 306 is, the more flexible the pedicle screw could be positioned within the bone anchor 100. In particular, the left inner face 146 and the right inner side 152 are aligned substantially parallel with the left leaning trajectory 302 for maximizing the trajectory range 306 for the left leaning trajectory 302. Similarly, the right inner face 186 and the left inner side 112 are also aligned substantially parallel with the right leaning trajectory 304 for maximizing the trajectory range 306 for the right leaning trajectory 304. In addition, the left inner face 146, the right inner side 152, the right inner face 186 and the left inner side 112 have smooth surfaces for not inhibiting movement of the pedicle screw along the left leaning trajectory 302 or the right leaning trajectory 304. The curved arrows in FIG. 6 show that the trajectory 300 is motivated to move by rotation of the adjustable head 200 in relation to the base 104 around the left hinge 142 and the right hinge 182.

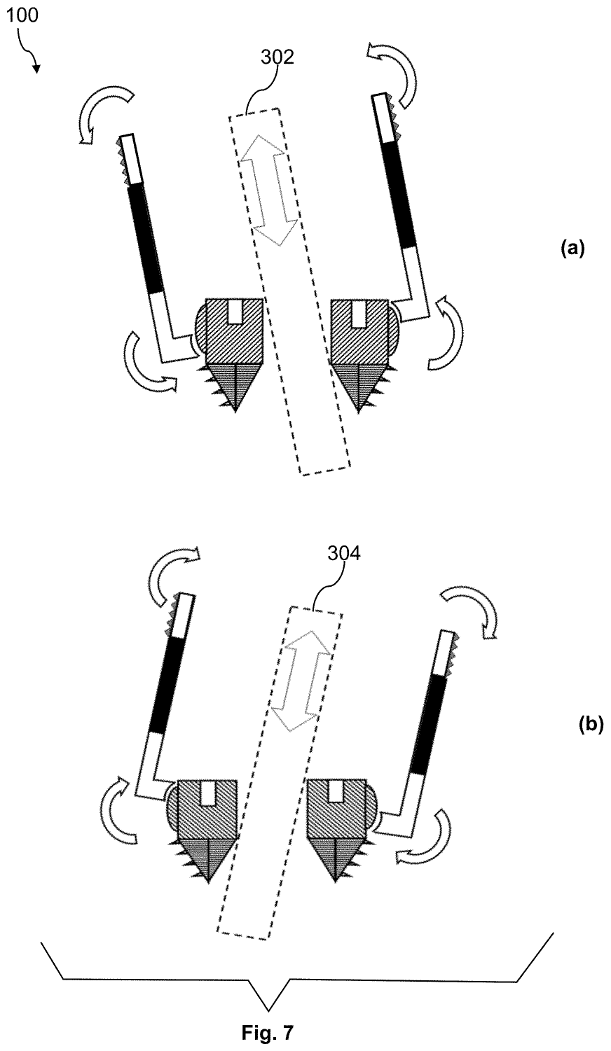

FIG. 7 illustrates (a) a cross-sectional view of the left leaning trajectory 302 when the bone anchor 100 is rotated to the left side (as indicated by the curved arrows); and (b) a cross-sectional view of the right leaning trajectory 304 when the bone anchor 100 is rotated to the right side (as indicated by the curved arrows).

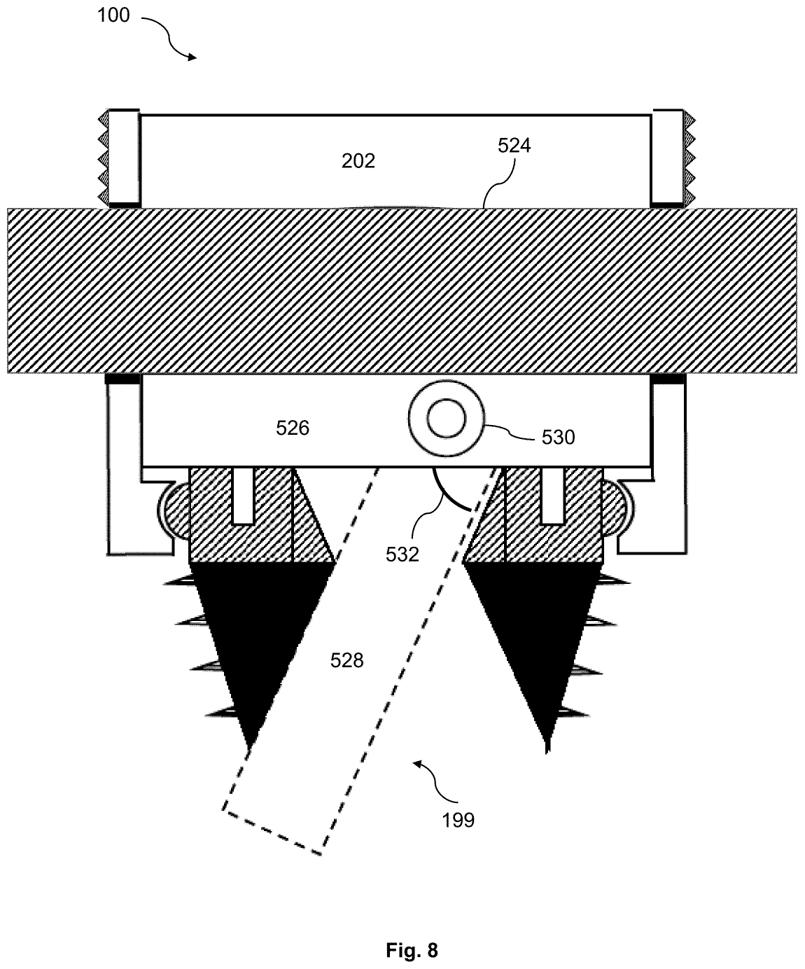

FIG. 8 illustrates a cross-sectional view of the bone anchor 100 loaded with a polyaxial screw 199. The polyaxial screw 199 has a screw base 526 loaded inside the interval cavity 202 and positioned below the bar 524. The polyaxial screw 199 is supported by the base 104. The polyaxial screw 199 also has a screw shaft 528 (as indicated in dash square) extending into the aperture 106. In particular, the screw shaft 528 is movably coupled to the screw base 526 via a polyaxial joint 530 for moving the polyaxial screw 199 towards multiple axes; and thereby enabling the screw shaft 528 to remain correctly aligned with the trajectory while simultaneously allowing the screw base 526 to remain flush with the base 104 of the bone anchor 100. In addition, the screw shaft 528 is set at an acute angle 532 relative to the screw base 526. A locking nut (not shown) may be superimposed on the screw base 526 for fixing the pedicle screw 199 in place.

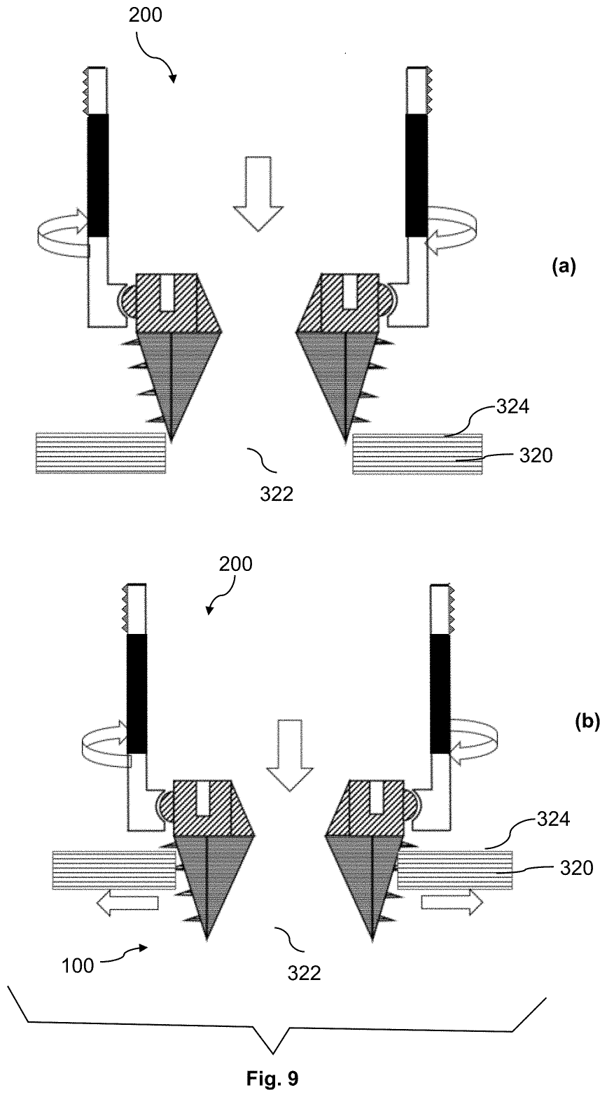

FIG. 9 illustrates (a) a cross-sectional view of the bone anchor 100 before purchase with surrounding cortex 320; and (b) a cross-sectional view of the bone anchor 100 after purchase with the surrounding cortex 320. In FIG. 9(a), the bone anchor 100 is motivated by rotating the adjustable head 200 to a pre-determined position within the decorticated facet 322 in the surrounding cortex 320. An external twisting force 324 (shown as a vertical arrow) is then applied to the adjustable head 200. In FIG. 9(b), the external twisting force 324 is transferred to the bone anchor 100 and results in tension to cortical walls 326 of the surrounding cortex 320. The tension would break the cortical wall 326 and squeeze the surrounding cortex 320 away from the decorticated facet 322 (shown as a horizontal arrow) for creating a space larger than the bone anchor 100 to be screwed into the decorticated facet 322, thereby providing better purchase with the surrounding cortex.

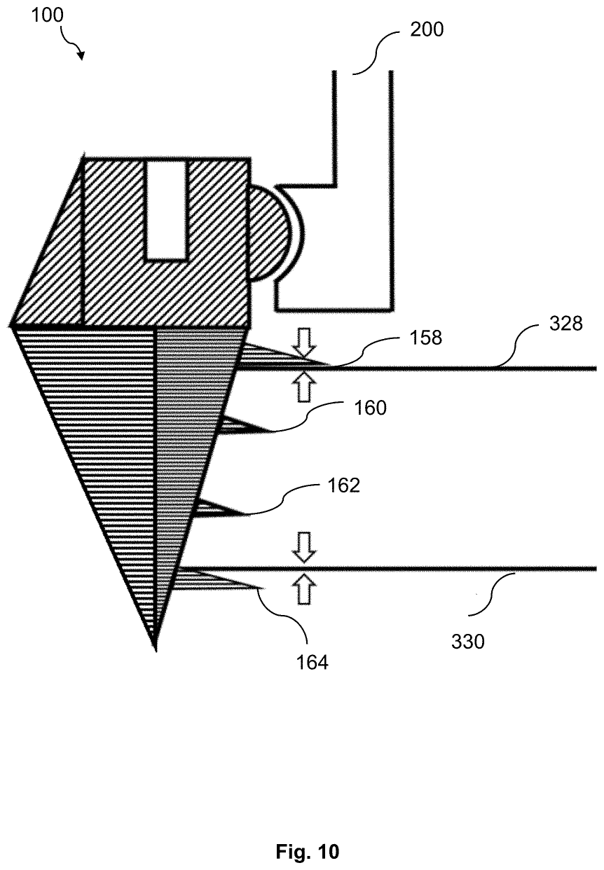

FIG. 10 illustrates a cross-sectional view of the bone anchor 100 secured within the surrounding cortex 320. The surrounding cortex 320 has an outer layer 328 and an inner layer 330. The screw threads 158-164 are secured with the surrounding cortex 320 between the outer layer 328 and the inner layer 330 for minimizing or even eliminating advancement and pullout of the bone anchor 100 from the surrounding cortex 320. There is minimal movement, i.e. no advancement nor pullout of bone anchor 100.

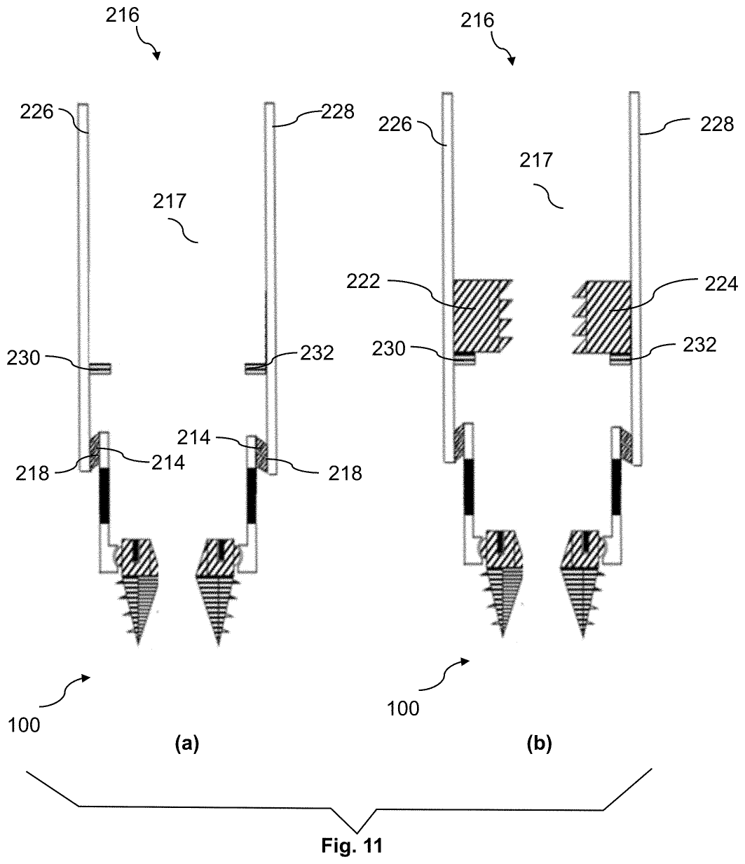

FIG. 11(a) illustrates a cross-sectional view of a flute 216 as the frame coupled with the adjustable head 200. The flute 216 has an external wall for forming an internal passage 217. In the cross-sectional view, the external wall is reduced to a left flute wall 226 and a right flute wall 228. The flute 216 has a complementary thread 218 matching the threaded end 214 of the adjustable head 200 such that the adjustable head 200 could extend or retract in relation to the flute 216. In addition, the flute 216 has an inset for supporting any loading into the flute 216. The inset has a ring structure; and thus a left inset 230 and a right inset 232 are shown in the cross-sectional view, which are attached to the left flute wall 226 and the right flute wall 228, respectively. FIG. 11(b) illustrates a cross-sectional view of the frame 214 having an internal stabilizing component 220. The internal stabilizing component 220 also has a ring structure; and thus a left internal stabilizing component 222 and a right internal stabilizing component 224 are shown in the cross-sectional view, which are attached to the left flute wall 226 and the right flute wall 228, respectively. The inset 230, 232 provide further stability to the stabilizing component 220 by preventing excessive downward force when the surgical instruments are inserted for preparing the pedicle tract and finally fixing the screw in the vertebral cortex.

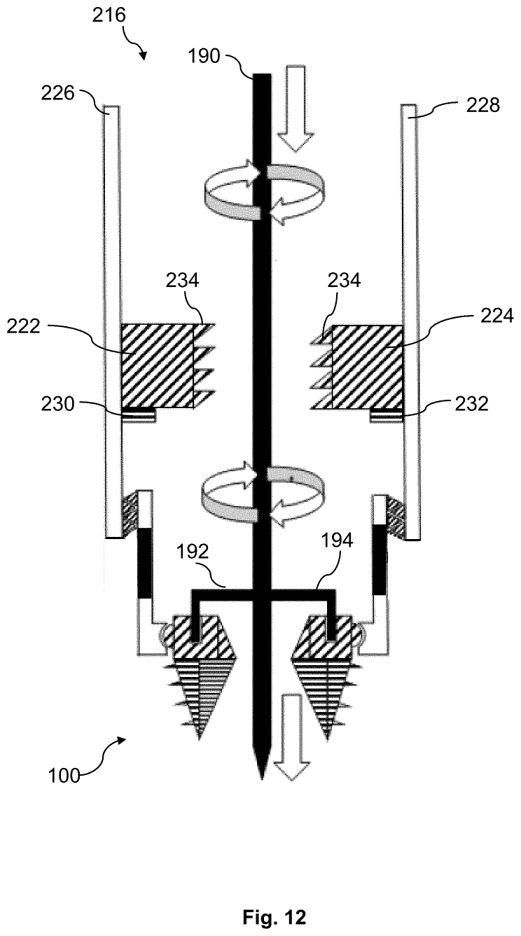

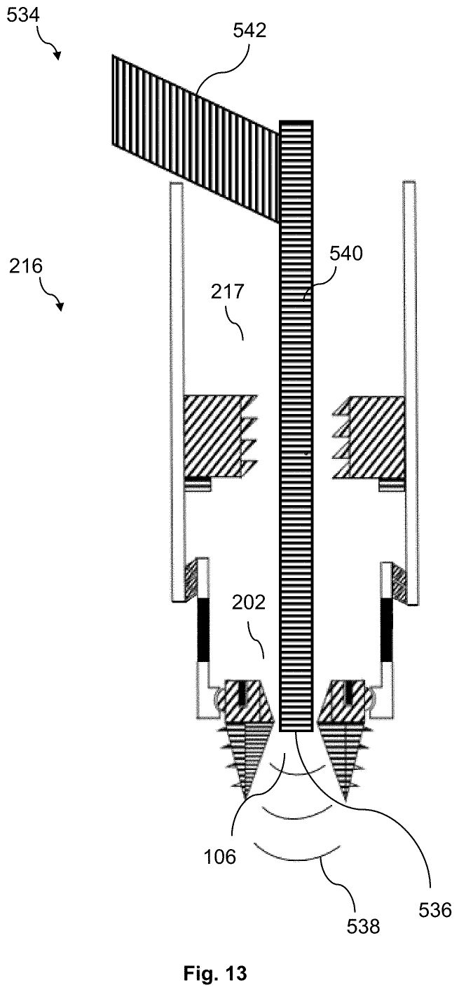

FIG. 12 to FIG. 16 illustrates cross-sectional views of an internal stabilizing component 220 to the bone anchor 100 in five subsequent stages of pedicle preparation and screw insertion processes. FIG. 12 shows a first stage when the bone anchor 100 is screwed into the decorticated facet 320, the stylet 190 and the stylet arms 192, 194 are inserted into the channel 202 for stabilizing the bone anchor 100. The bone anchor 100 is secured to the vertebral cortex by rotating (shown as rotating arrows) and meanwhile pushing downwardly (shown as vertical arrows) the stylet 190. The internal stabilizing component 220 has a plurality of (such as 4) stabilizing teeth facing to the stylet 190. FIG. 13 shows a second stage when an ultrasonic device 534 passing through the internal passage 217 of the flute 216. The ultrasonic device 534 has an ultrasonic transducer 536 for firstly generating ultrasound waves 538 in the ultrasonic range and then receiving echoes of the ultrasound waves 538. In this way, the vertebral cortex surrounding the ultrasonic transducer 536 is detected. The ultrasonic transducer 536 is coupled to an ultrasonic shaft 540 of the ultrasonic device 534 such that the ultrasonic transducer 536 is placed into the aperture 106 by inserting the ultrasonic shaft 540 through the internal passage 217 of the flute 216 and the internal cavity 202 of the adjustable head 200. The ultrasonic shaft 540 is further coupled to an ultrasonic handle 542 for manipulating the ultrasonic device 534.

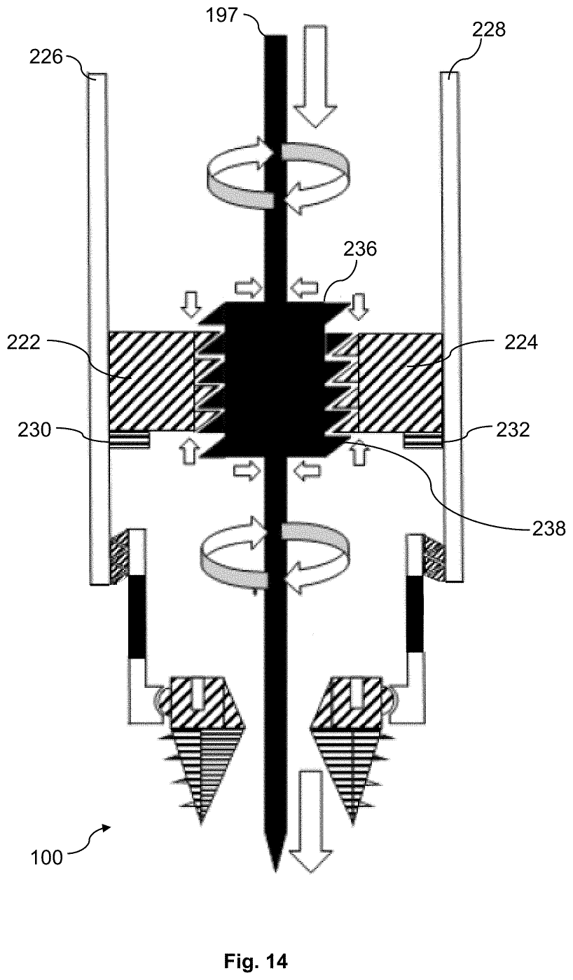

FIG. 14 shows a third stage when a pedicle or awl 197 of spinal instrument is used to prepare a pedicle tract. A threaded bolt 236 is attached onto the pedicle or awl 197 and positioned exactly within the internal stabilizing component 220. In the cross-sectional view, the threaded bolt 236 is matched in-between the left internal stabilizing component 222 and the right internal stabilizing component 224. In particular, the threaded bolt 236 has multiple bolt teeth 238 which are complementary to the stabilizing teeth 234 of the internal stabilizing component 220 such that the threaded bolt 236 bites the left internal stabilizing component 222 and the right internal stabilizing component 224. The pedicle or awl 197 can rotate and move downwardly relative to the fixed internal stabilizing component 220.

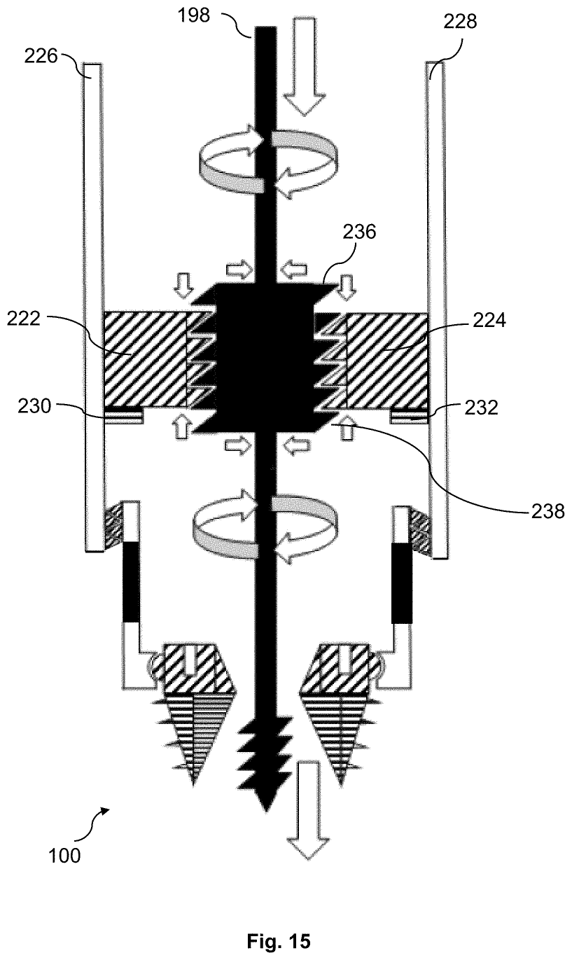

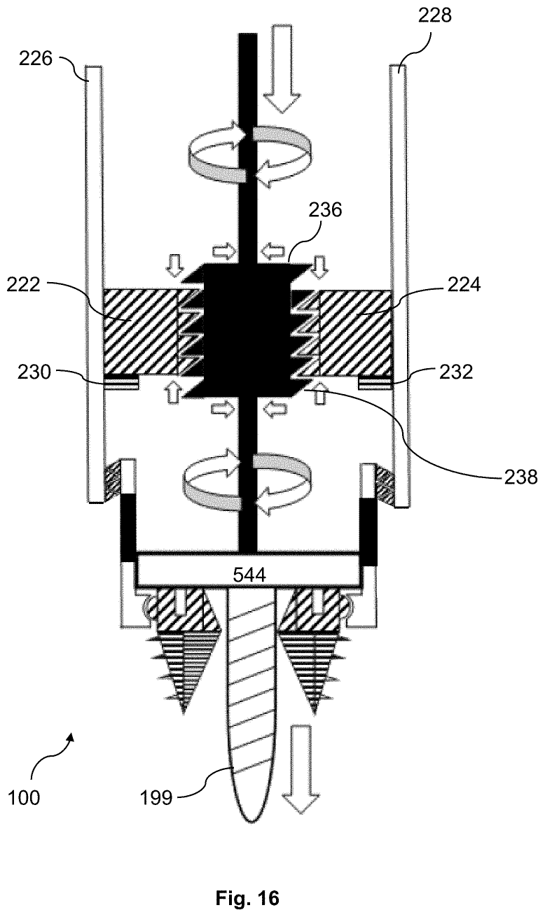

FIG. 15 shows a fourth stage when a tap screw 198 is used to ream the pedicle in preparation for the pedicle screw. The tap screw 198 is attached near the sharp end 196 for reaming the pedicle. Similarly, the threaded bolt 236 is also attached onto the tap screw 198 for matching with the internal stabilizing component 220. In the cross-sectional view, the threaded bolt 236 is positioned exactly in-between the left internal stabilizing component 222 and the right internal stabilizing component 224. FIG. 16 shows a following fifth stage when the polyaxial screw 199 is screwed through the bone anchor 100 until a polyaxial screw base 544 of the polyaxial screw 199 lies flush with the base 104 of the bone anchor 100. The polyaxial configuration of the polyaxial screw 199 preserves flexibility for a shaft of the polyaxial screw 199 to align with the intended trajectory while simultaneously ensuring the polyaxial screw base 544 is flush with the base 104. The bar 524 for interconnecting multiple bone anchors 100 from adjacent levels on the same side of the spine is superimposed on the polyaxial screw 199, before the polyaxial screw 199 is further firmly secured by a locking nut. Similarly, the threaded bolt 236 is also attached onto the polyaxial screw 199 for matching with the internal stabilizing component 220. In the cross-sectional view, the threaded bolt 236 is positioned exactly in-between the left internal stabilizing component 222 and the right internal stabilizing component 224.

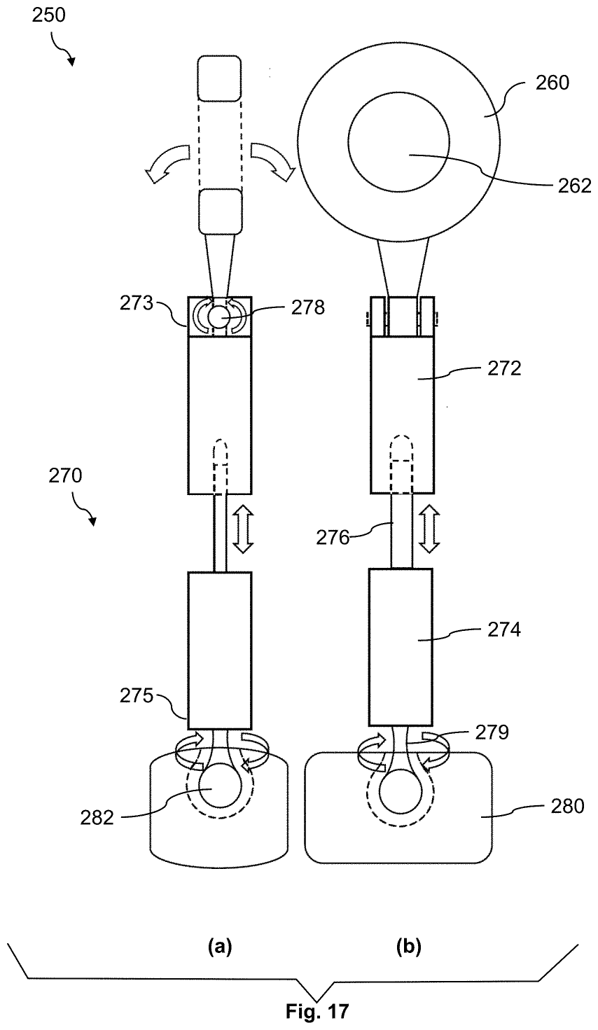

FIG. 17 illustrates (a) a cross-sectional view and (b) an aerial view of a linking mechanism 250. The linking mechanism 250 comprises a circular frame 260, an external arm 270 and a cuboidal clamp 280 movably coupled in sequence. The circular frame 260 has a cavity 262 for coupling the flute 216. The external arm 270 has a first sub-arm 272 and a second sub-arm 274 adjoined by a sliding hinge 276. The sliding hinge 270 allows extension and contraction of the first sub-arm 272 and the second sub-arm 274 along a single axis. The straight arrows show the extension and contraction. The external arm 270 movably couples the circular frame 260 at a proximal end 273 of the first sub-arm 272 and the cuboidal clamp 280 at a distal end 275 of the second sub-arm 274. In particular, the first sub-arm 272 has a rotation axis 278 at the proximal end 273 and thus the circular frame 260 can rotate around the rotation axis 278. While the second sub-arm 274 has a rotation rope 279 at the distal end 275 and thus the external arm 270 can rotate around the cuboidal clamp 280. The cuboidal clamp 280 has a ball and socket joint 282 working in conjunction with the rotation rope 279 for allowing the external arm 270 to freely rotate around the cuboidal clamp 280.

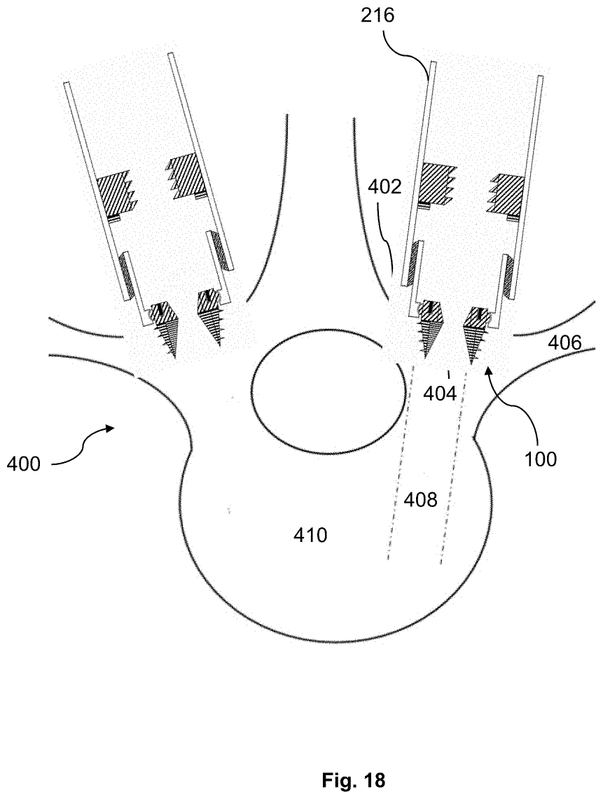

FIG. 18 illustrates a cross-sectional view of two bone anchors 100 secured to vertebra cortex 400. The vertebra cortex has lamina 402, shaved facet 404, transverse process 406, pedicle 408 and vertebral body 410. The bone anchors 100 are secured to the vertebra 400 by screwing the bone anchors 100 into the decorticated facet/inferior articular process/lateral mass 404 until they are in close proximity to the pedicle 408.

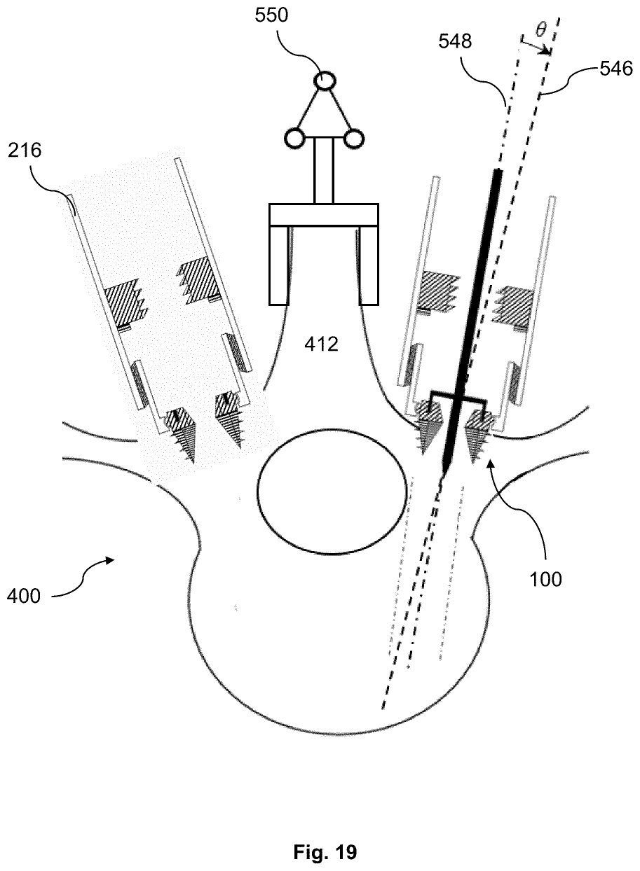

FIG. 19 illustrates a cross-sectional view of adjustment of real-time trajectory 548 to coincide with planned trajectory 546. A theta Θ is presented in FIG. 19 to show misalignment of the real-time trajectory 548 to the planned trajectory 546. The real-time trajectory 548 is then adjusted until the theta Θ becomes zero, which means the real-time trajectory 548 actually coincide with planned trajectory 546. In addition, a reference frame 550 is loaded at another vertebra cortex adjacent to the vertebra cortex to initially guide the real-time trajectory 548.

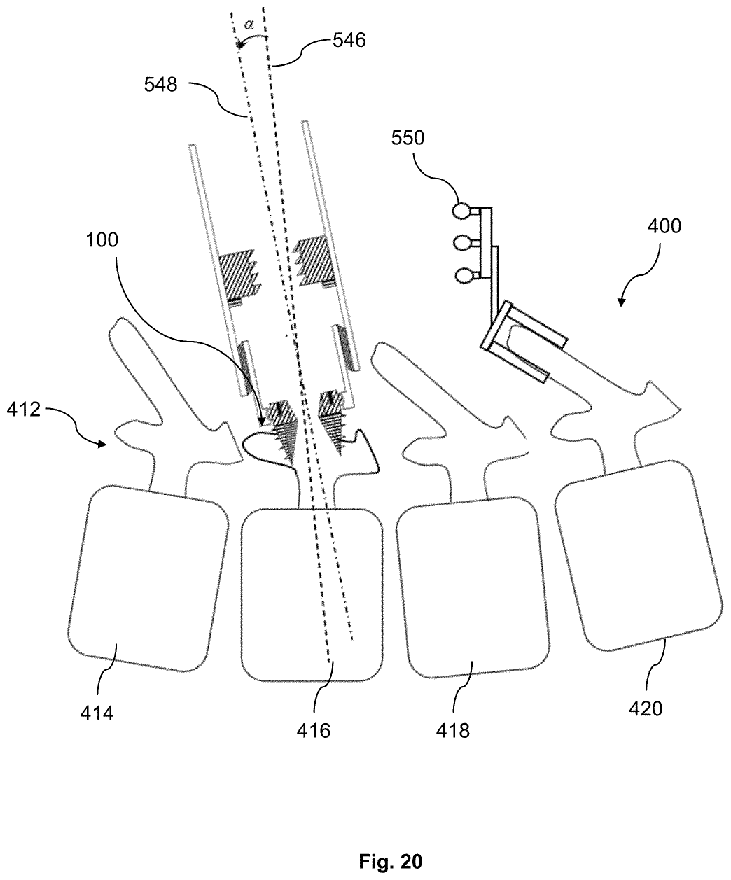

FIG. 20 illustrates a lateral view of the adjustment of the real-time trajectory 548 to coincide with the planned trajectory 546. A spinal column 412 has multiple vertebras, as such a first vertebra 414, a second vertebra 416, a third vertebra 418 and a fourth vertebra 420 as shown in the FIG. 20. Before a surgical operation would be performed on the second vertebra 416, the reference frame 550 is loaded at the fourth vertebra 420 for guiding the real-time trajectory 548 to the second vertebra 416. During the surgical operation, the real-time trajectory 548 is adjusted to coincide the planned trajectory 546 until the theta Θ becomes zero.

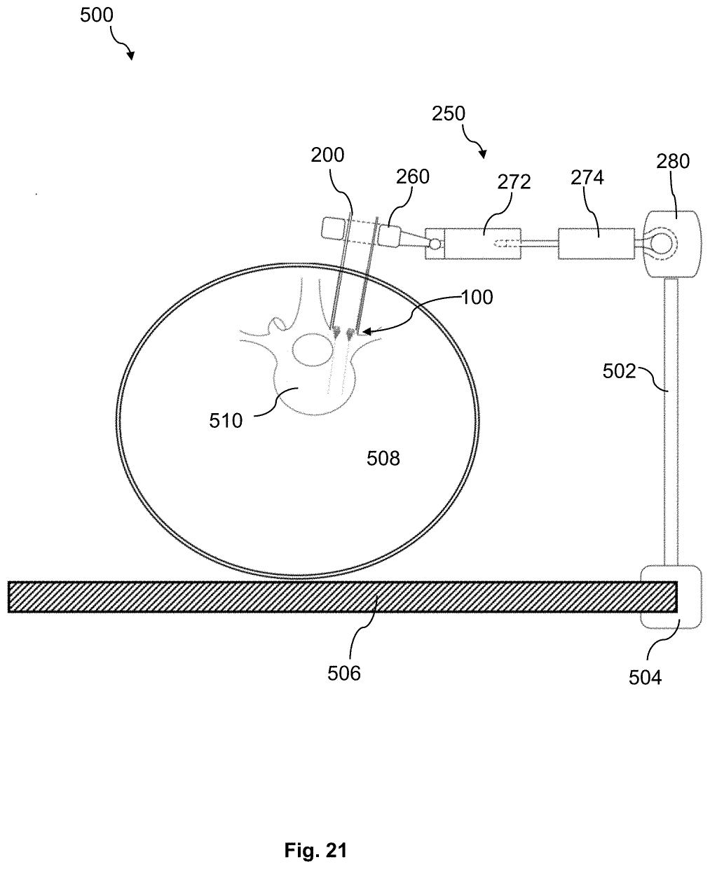

FIG. 21 illustrates a cross-sectional view of an external clamping mechanism 500 for stabilizing the bone anchor 100 to a specific vertebra 510 within the trunk (such as a thorax or abdomen) 508. The external clamping mechanism 500 has a rod 502 as a supporting means movably coupled to the cuboidal clamp 280 and a table clamp 504 coupled to the rod 502 at one end and a surgical table 506 on the other end. The trunk 508 is fixed on the surgical table 506 during surgery. As a result, the bone anchor 100 is stabilized to the trunk 508.

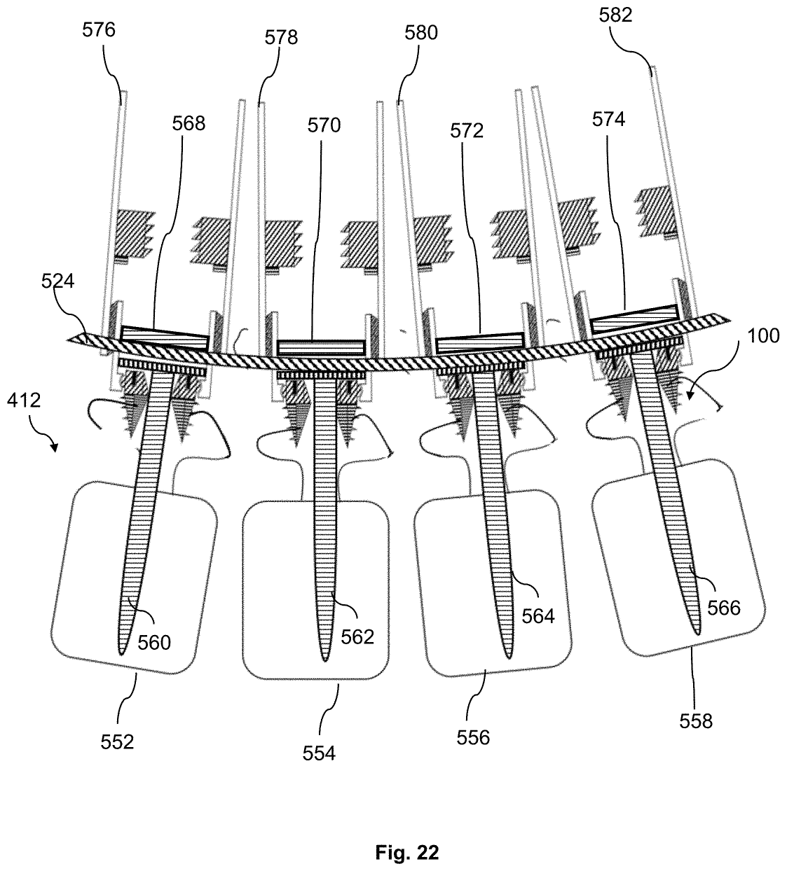

FIG. 22 illustrates a cross-sectional view of a series of bone anchors 100 on a series of vertebras. As shown in FIG. 22, a first bone anchor 552, a second bone anchor 554, a third bone anchor 556 and a fourth bone anchor 558 are loaded into the first vertebra 414, the second vertebra 416, a third vertebra 418 and the fourth vertebra 420 for fixing a first pedicle screw 560, a second pedicle screw 562, a third pedicle screw 564 and a fourth pedicle screw 566, respectively. A series of flutes 216, i.e. a first flute 576, a second flute 578, a third flute 580 and a fourth flute 582 are assembled to the first bone anchor 552, the second bone anchor 554, the third bone anchor 556 and the fourth bone anchor 558, respectively. The bar 524 is used to connect the bone anchors 552-556 together in sequence. To conform to the curved profile of the spinal column 412, the bar 524 also has a curved configuration accordingly. In addition, a series of locking nuts, i.e. a first locking nut 568, a second locking nut 570, a third locking nut 572 and a fourth locking nut 574 are loaded onto the bar 524 for fixing the bone anchors 552-556 more firmly in place.

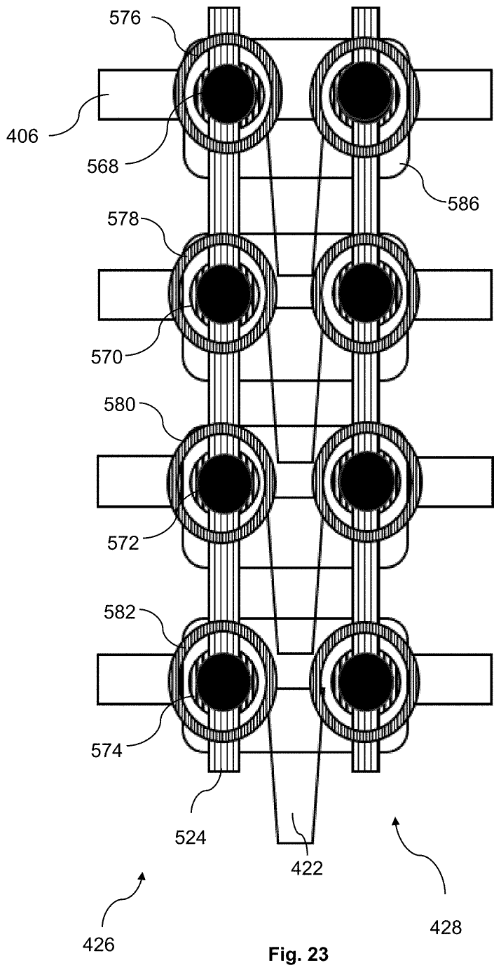

FIG. 23 illustrates an aerial view of the series of bone anchors 100 connected together by the bar 524. As shown in FIG. 23, the series of bone anchors 552-558 and locking nuts 568-574 are loaded onto a series of vertebrae 414-420 in a first spinal line 426. Similarly, four bone anchors 100 and four locking nuts 568-574 are loaded onto the same four vertebrae in a second spinal line 428. The first spinal line 426 and the second spinal line 428 are separated by a spinous process 428. In other words, two bone anchors 100 and two locking nuts are loaded onto the same vertebra.

In the application, unless specified otherwise, the terms “comprising”, “comprise”, and grammatical variants thereof, intended to represent “open” or “inclusive” language such that they include recited elements but also permit inclusion of additional, non-explicitly recited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. The description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### REFERENCE NUMERALS

100 bone anchor;
102 conical tapered screw;
104 base/central body/main body;
106 aperture;
108 vertical direction;
110 left portion;
112 left inner side;
114 left outer side;
116 left sharp point;
118 first left screw tip;
120 second left screw tip;
122 third left screw tip;
124 fourth left screw tip;
126 first left distance;
128 second left distance;
130 third left distance;
132 left inner angle;
134 left outer angle;
140 left portion of the base;
142 left hinge;
144 left hole;
146 left inner face;
150 right portion;
152 right inner side;
154 right outer side;
156 right sharp point;
158 first right screw tip;
160 second right screw tip;
162 third right screw tip;
164 fourth right screw tip;
166 first right distance;
168 second right distance;
170 third right distance;
172 right inner angle;
174 right outer angle;
180 right portion of the base;
182 right hinge;
184 right hole;
186 right inner face;
190 stylet;
192 first stylet arm;
194 second stylet arm;
196 sharp end;
197 pedicle or awl;
198 tap screw;
199 polyaxial screw;
200 adjustable head;
202 internal cavity;
204 left concave structure;
206 right concave structure;
208 side aperture;
210 left aperture;
212 right aperture;
214 threaded end;
216 flute;
217 internal passage;
218 complementary thread;
220 internal stabilizing component;
222 left internal stabilizing component;
224 right internal stabilizing component;
226 left flute wall;
228 right flute wall;
230 left inset;
232 right inset;
234 stabilizing teeth (internal stabilizing component);
236 threaded bolt;
238 bolt teeth (threaded bolt of spinal instrument);
250 linking mechanism;
260 circular frame;
262 cavity;
270 external arm;
272 first sub-arm;
273 proximal end
274 second sub-arm;
275 distal end
276 sliding hinge;
278 rotation axis;
279 rotation rope;
280 cuboidal clamp;
282 ball and socket joint;
300 trajectory;
302 left leaning trajectory;
304 right leaning trajectory;
306 trajectory range;
320 surrounding cortex;
322 decorticated facet;
324 external twisting force;
326 cortical wall of the surrounding cortex;
328 outer layer of the surrounding cortex;
330 inner layer of the surrounding cortex;
400 vertebral cortex;
402 lamina;
404 shaved facet;
406 transverse process;
408 pedicle;
410 vertebral body;
412 spinal column;
414 first vertebra;
416 second vertebra;
418 third vertebra;
420 fourth vertebra;
422 spinous process;
424 vertebral bod;
426 first spinal line;
428 second spinal line;
500 pedicle tract stabilization system;
502 rod;
504 table clamp;
506 surgical table;
508 trunk;
510 vertebra;
512 conical cutting profile;
514 upper cutting line;
516 lower cutting line;
518 distance;
520 planned trajectory;
522 trajectory angle;
524 bar;
526 screw base;
528 screw shaft;
530 polyaxial joint;
532 acute angle;
534 ultrasonic device;
536 ultrasonic transducer;
538 ultrasound waves;
540 ultrasonic shaft;
542 ultrasonic handle;
544 polyaxial screw base;
546 planned trajectory;
548 real-time trajectory;
550 reference frame;
552 first bone anchor;
554 second bone anchor;
556 third bone anchor;
558 fourth bone anchor;
560 first pedicle screw;
562 second pedicle screw;
564 third pedicle screw;
566 fourth pedicle screw;
568 first locking nut;
570 second locking nut;
572 third locking nut;
574 fourth locking nut;
576 first flute;
578 second flute;
580 third flute;
582 fourth flute;
584 first level;
586 vertebral body;
600 crown tapered screw;
602 crown cutting profile;
604 left portion;
606 right portion;
608 middle portion;
610 upper cutting line;
612 middle cutting line;
614 bottom cutting line;
616 first distance;
618 second distance;
620 first planned trajectory;
621 second planned trajectory;
622 trajectory angle;

While non-limiting exemplary embodiment(s) has/have been described with respect to certain specific embodiment(s), it will be appreciated that many modifications and changes may be made by those of ordinary skill in the relevant art(s) without departing from the true spirit and scope of the present disclosure. It is intended, therefore, by the appended claims to cover all such modifications and changes that fall within the true spirit and scope of the present disclosure. In particular, with respect to the above description, it is to be realized that the optimum dimensional relationships for the parts of the non-limiting exemplary embodiment(s) may include variations in size, materials, shape, form, function and manner of operation.

The Abstract of the Disclosure is provided to comply with 37 C.F.R. § 1.72(b) and is submitted with the understanding that it will not be used to interpret or limit the scope or meaning of the claims. In addition, in the above Detailed Description, various features may have been grouped together or described in a single embodiment for the purpose of streamlining the disclosure. This disclosure is not to be interpreted as reflecting an intention that the claimed embodiment(s) require more features than are expressly recited in each claim. Rather, as the following claims reflect, inventive subject matter may be directed to less than all of the features of any of the disclosed non-limiting exemplary embodiment(s). Thus, the following claims are incorporated into the Detailed Description, with each claim standing on its own as defining separately claimed subject matter.

The above disclosed subject matter is to be considered illustrative, and not restrictive, and the appended claims are intended to cover all such modifications, enhancements, and other embodiment(s) which fall within the true spirit and scope of the present disclosure. Thus, to the maximum extent allowed by law, the scope of the present disclosure is to be determined by the broadest permissible interpretation of the following claims and their equivalents, and shall not be restricted or limited by the above detailed description.

What is claimed as new and what is desired to secure by Letters Patent of the United States is:

1. A bone anchor for stabilizing vertebra, comprising:
a gripping mechanism for securing the bone anchor to the vertebra; and
a central body for coupling the gripping mechanism;
wherein the gripping mechanism is configured to form an aperture for a pedicle screw to pass through.

2. The bone anchor of claim 1, wherein the gripping mechanism comprises:
a tapered screw with a plurality of screw threads for securing to vertebra cortex surrounding the vertebra.

3. The bone anchor of claim 2, wherein the tapered screw has an inner side with an inner angle and an outer side with an outer angle, wherein the inner angle is larger than the outer angle.

4. The bone anchor of claim 3, wherein the inner side and the outer side of the tapered screw are configured to form a sharp edge for cutting through the vertebra cortex.

5. The bone anchor of claim 4, wherein the central body comprises:
a base coupled to the tapered screw, wherein the base is opposed to the sharp edge.

6. The bone anchor of claim 5, further comprising:
an adjustable head movably coupled to the base, wherein the adjustable head is configured to form an internal cavity for the pedicle screw to pass through.

7. The bone anchor of claim 6, wherein the bone anchor is configured to form a screw trajectory, wherein the screw trajectory is adjusted in a certain range in the internal cavity and the aperture.

8. The bone anchor of claim 6, wherein the adjustable head is configured to form a side aperture for a bar to laterally pass through.

9. The bone anchor of claim 6, wherein the adjustable head comprises:
a threaded end for coupling to a fixation mechanism.

10. The bone anchor of claim 5, further comprising:
a stylet coupled to the base for stabilizing the bone anchor when the bone anchor is screwed into decorticated facet in the vertebrae vortex.

11. The bone anchor of claim 10, wherein the stylet comprises:
a first stylet arm and a second stylet coupled to a left side and a right side of the base, respectively.

12. The bone anchor of claim 8, further comprising:
a locking nut superimposed onto the bar for securing the adjustable head in place.

13. The bone anchor of claim 7, wherein the pedicle screw comprises:
a polyaxial screw, wherein the polyaxial screw is easily aligned correctly with the screw trajectory.

14. The bone anchor of claim 13, wherein the polyaxial screw further comprises:
a polyaxial screw base configured to load on the base; and
a screw shaft movably coupled to the screw chassis; wherein the screw shaft is configured to form an acute angle with the screw base.

15. A fixation mechanism for fixing a bone anchor, comprising:
a frame coupled to the bone anchor, wherein the frame is configured to form an internal passage for a pedicle screw to pass through.

16. The fixation mechanism of claim 15, further comprising:
an internal stabilizing component coupled within the frame for minimizing buckling of the bone anchor in operation.

17. The fixation mechanism of claim 16, further comprising:
an inset coupled within the frame for supporting the internal stabilizing component.

18. The fixation mechanism of claim 17, wherein the internal stabilizing component has a plurality of stabilizing teeth complementary to bolt threads of a cutting instrument.

19. A pedicle tract stabilization system for preparing and placing a pedicle screw in vertebral cortex, comprising:
at least one bone anchor for stabilizing at least one vertebra; and
a fixation mechanism movably attached to the at least one bone anchor;
wherein the at least one bone anchor and the fixation mechanism are configured to form a through channel for a pedicle screw to pass through.

20. The pedicle tract stabilization system of claim 19, further comprising:
an external clamping mechanism for clamping the pedicle tract stabilizing system to a stationary object; and
a linking mechanism for movably coupling the fixation mechanism and the external clamping mechanism.

21. The pedicle tract stabilization system of claim 20, wherein the linking mechanism further comprises:
a circular frame coupled to the fixation mechanism;
an external arm having a proximal end and a distal end, wherein the circular frame is movably coupled to the proximal end; and
a cuboidal clamp movably coupled to the distal end and the external clamping mechanism.

22. The pedicle tract stabilization system of claim 21, wherein the external arm further comprises:
a first sub-arm and a second sub-arm adjoined by a sliding-hinge mechanism.

23. The pedicle tract stabilization system of claim 21, wherein the cuboidal clamp further comprises:
a ball and a socket joint movably coupled together for providing a flexible maneuverability.

24. The pedicle tract stabilization system of claim 20, wherein the external clamping mechanism further comprises:
a supporting means movably coupled to the cuboidal clamp; and
a clamping means coupled to the supporting means and the stationary object.

25. The pedicle tract stabilization system of claim 24, further comprising:
a reference frame for locating the at least one bone anchor to a targeted vertebra.
